(19) United States
(12) Patent Application Publication
(10) Pub. No.: US 2024/0108649 A1
(43) Pub. Date: Apr. 4, 2024

(54) GUMMY DOSAGE FORMS

(71) Applicant: JOHNSON & JOHNSON CONSUMER INC., Skillman, NJ (US)

(72) Inventors: Michel R. IGLESIAS, Lindon, UT (US); Gregory C. ANDERSON, Lansdale, PA (US); Timothy Douglas KUTCH, Phoenixville, PA (US); Isha B. SHAH, Troy, NY (US)

(21) Appl. No.: 18/475,416

(22) Filed: Sep. 27, 2023

Related U.S. Application Data
(60) Provisional application No. 63/377,781, filed on Sep. 30, 2022.

(51) Int. Cl. A61K 33/06; A61K 9/00; A61K 36/53; A61K 47/12; A61K 47/26; A61K 47/36

(52) U.S. Cl. CPC A61K 33/06; A61K 9/0056; A61K 36/53; A61K 47/12; A61K 47/26; A61K 47/36

(57) ABSTRACT

Described are gummy dosage forms comprising tricalcium phosphate and a relaxing ingredient selected from Lemon balm extract, L-theanine, GABA, or mixtures thereof.

### CROSS-REFERENCE TO RELATED APPLICATIONS

This application claims the benefit of provisional application U.S. 63/377,781 filed Sep. 30, 2022, the complete disclosure of which is hereby incorporated by reference in its entirety herein for all purposes.

### FIELD

The present disclosure relates to gummy dosage forms comprising tricalcium phosphate and a relaxing ingredient and processes of manufacturing the same. The present disclosure further relates to a method of treating a subject suffering from a gastrointestinal tract condition, for example, a gastric disease or disorder, using such gummy dosage forms.

### BACKGROUND

Gummy dosage forms are known for their convenience and versatility in delivering therapeutic ingredients in the medicinal, nutritional, and natural space. They have been utilized to deliver a wide variety of ingredients such as pain treatment ingredients, digestive health treatment ingredients, cough-cold treatment ingredients, nutritional supplements, and natural or herbal ingredients. In certain cases, gummy dosage forms are known as an alternative delivery format for consumers who cannot easily swallow pills, capsules, or tablets, or for consumers who do not prefer medicinal liquids.

It is known that exposure to stress, especially chronic stress, is a major risk factor in the pathogenesis of different diseases of the gastrointestinal (GI) tract. Stress and depression are related to different digestive disorders, and they may be predisposing factors for irritable bowel syndrome (IBS), reflux esophagitis, and other functional GI disorders.

Lemon balm, or Melissa officinalis, is a plant from the mint family with a number of phytochemicals including phenolic acids, terpenes, rosmarinic and caffeic acids, eugenol acetate, and tannins. The European Scientific Cooperative on Phytotherapy (ESCOP) has indicated the use of lemon balm internally for tenseness, restlessness and irritability. Recently, a double-blind placebo-controlled clinical trial with 80 patients with chronic stable angina taking 3 g/day of M. officinalis supplementation (capsules) for 8 weeks showed positive effects on stress and anxiety among other self-reported outcomes.

L-theanine is a non-protein amino acid mainly found naturally in green tea (Camellia sinensis) and some mushrooms (Boletus badius). It is a naturally occurring structural analogue of glutamate, L-theanine competes for the receptors with glutamate and is able to pass the blood-brain barrier to exert its relaxation effect. According to Health Canada theanine monograph, L-theanine helps to temporarily promote relaxation at doses of 200-250 mg/day. This is in line with a recent systematic review on the effects of L-theanine consumption and its ability to manage stress and anxiety levels where a dose of 200-400 mg/day was suggested.

GABA, or gamma-aminobutyric acid, is a non-proteinogenic amino acid that is present in bacteria, plants, and vertebrates. It functions is as the primary inhibitory neurotransmitter for the central nervous system. Low levels of GABA or impaired GABA functioning is associated with the etiology and maintenance of acute and chronic stress.

It is also known to bring rapid relief from the symptoms of excess stomach acidity by neutralization such as with an antacid.

Antacids used today are made from a variety of inorganic salts such as calcium carbonate, sodium bicarbonate, magnesium salts and aluminum salts. Magnesium hydroxide and aluminum hydroxide are the most potent magnesium and aluminum salts and are often used in combination. In addition, magnesium oxide, magnesium carbonate, aluminum phosphate, magaldrate, magnesium trisilicate, and aluminum sucrose sulfate (sucralfate) are also employed.

Accordingly, as there has been ongoing consumer demand for more treatment of digestive tract disorder such as inflammation and heartburn, there is a need to propose a dosage form acceptable for the consumer, such as a gummy dosage form, that can provide both an antiacid effect and an anti-stress effect. The presently disclosed subject matter addresses these and other needs.

### SUMMARY

The presently disclosed subject matter provides improved gummy dosage form. The gummy dosage form comprises tricalcium phosphate and a relaxing ingredient selected from Lemon balm extract, L-theanine, GABA or mixtures thereof.

In certain embodiments the relaxing ingredient can be Lemon balm extract.

In certain embodiments the gummy dosage form can comprise Lemon balm extract in an amount of from about 10 mg to about 2 g.

In certain embodiments the relaxing ingredient can be L-theanine.

In certain embodiments the gummy dosage form can comprise L-theanine in an amount of from about 10 mg to about 2 g.

In certain embodiments the relaxing ingredient can be GABA.

In certain embodiments the gummy dosage form can comprise GABA in an amount of from about 10 mg to about 800 mg.

In certain embodiments the gummy dosage form can comprise tricalcium phosphate in an amount of from about 100 mg to about 2 g.

In certain embodiments the gummy dosage form can comprise sucrose.

In certain embodiments the gummy dosage form can be substantially free of one or more of high fructose corn syrup, artificial sweeteners, artificial flavors, gluten, or gelatin.

In certain embodiments the gummy dosage form can have a mass of from about 1 gram to about 8 grams.

In certain embodiments the gummy dosage form can have a mass of from about 4 grams to about 6 grams.

The present disclosure further provides a method of making the gummy dosage form according to the present disclosure, comprising the steps of: a) blending the tricalcium phosphate and the relaxing ingredient in an aqueous pectin solution, b) pouring the blend in a mold, and c) allowing the blend to cool in the mold to form the gummy dosage form.

The present disclosure further provides the gummy dosage form for use in a method of treating a gastric disease or disorder.

The present disclosure further provides a method of treatment of a subject suffering from difficulty of sleeping related to a gastric disease or disorder.

### DETAILED DESCRIPTION

The presently disclosed subject matter relates to improved gummy dosage forms including tricalcium phosphate, an ingredient indicated as an antiacid for the treatment of gastric diseases or disorder such as heartburn. The improved gummy dosage forms also include a relaxing ingredient selected from Lemon balm extract, L-theanine, GABA or their mixtures. Without being bound by theory, it is believed that the inclusion of a relaxing ingredient may have an effect on a patient's stress level and, thus, may reduce the predisposition to gastric diseases or other disorders such as heartburn. The relaxing ingredient may also reduce a patient's inability or difficulty to fall asleep that may be related to gastric diseases or disorder such as heartburn.

Gummy dosage forms of the present disclosure and methods of making the same advantageously ensure a combined antiacid and relaxing ingredient effect to provide relief and treatment to consumers suffering from gastric diseases or disorder such as heartburn, while offering the convenient and pleasant format of a gummy. Additionally, the combined antiacid and relaxing ingredient effect can provide relief and treatment to consumers having difficulties falling asleep due to gastric diseases or disorder such as heartburn.

Methods of using gummy dosage forms as disclosed herein are further provided and include the treatment of a subject suffering from gastric disease or disorder, such as dyspepsia, abdominal fullness, heartburn, nausea, belching, or upper abdominal pain. People may also experience feeling full earlier than expected when eating. Dyspepsia is a common problem and is frequently caused by gastroesophageal reflux disease (GERD) or gastritis. In certain embodiments, the subject can be suffering from difficulty sleeping from the gastric disease or disorder, particularly from the heartburn.

These and other aspects of the disclosed subject matter are discussed in more detail below and in the Examples. For clarity and not by way of limitation, this detailed description is divided into the following sub-portions: Definitions; Gummy Dosage Forms; Methods of Making Gummy Dosage Forms; Methods of Using Gummy Dosage Forms; Features of Gummy Dosage Forms; and Other Dosage Forms.

### Definitions

The terms used in this specification generally have their ordinary meanings in the art within the context of this disclosure and in specific context where each term is used. Certain terms are discussed below, or elsewhere in the specification, to provide additional guidance in describing the compositions and methods of the disclosure and how to make and use them.

As used in the specification and the appended claims, the singular forms “a,” “an,” and “the” include the plural referents unless the context clearly dictates otherwise. Thus, for example, reference to “an ingredient” includes mixtures of ingredients.

As used herein, the terms “about” or “approximately” mean within an acceptable error range for the particular value as determined by one of ordinary skill in the art, which will depend in part on how the value is measured or determined (i.e., the limitations of the measurement system). For example, “about” can mean within three or more standard deviations per practice in the art. Alternatively, “about” can mean a range of up to 10%, up to 5%, up to 3%, up to 1%, or up to 0.5% of a given value. Particularly with respect to systems or processes, the term can mean within an order of magnitude, for example, within five-fold or within two-fold of a value.

As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

As used herein, the terms “dosage,” “dosage form,” “dose unit,” or “dose” refers to the amount of a therapeutic ingredient comprising therapeutically active agent(s) administered at a time. The terms “dosage,” “dosage form,” “dose unit,” or “dose” includes administration of one or more units of therapeutic ingredient administered at the same time. A therapeutic ingredient can include, for example, a pharmaceutical, natural, supplement or herbal ingredient.

As used herein, the term “gelling matrix” refers to the portion of the gummy dosage form wherein the gelling agents are hydrated, deposited and formed, including the bound water.

As used herein, the terms “gummy form” or “gummy dosage form” refers to a dosage form which retains its integrity and texture upon chewing, does not break into discrete, solid particulates upon chewing, utilizes a gelling matrix, and is intended to be swallowed. The gummy form or gummy dosage form can be palatable, edible, and can be similar in texture to confectionery jelly beans.

As used herein, the terms “% w/w” or “weight percent” refers to the percentage of an ingredient(s)/the total percentage by weight of the composition (100%). The terms “% w/w” or “weight percent” refer to the quantity by weight of a constituent or component, for example, in a gummy dosage form as a percentage of the overall weight of the gummy dosage form. The terms “weight percent”, “wt-%”, “wt. %”, and “wt %” are used interchangeably.

As used herein, the term “substantially free” refers to the quantity by weight of a constituent or component, for example, in a gummy dosage form; the weight percent of the said constituent or component being inferior to or less than 1%, or 0.5%, or 0.1%, or 0.05%, or 0.001% of the overall weight of the gummy dosage form.

### Gummy Dosage Forms

Gummy dosage forms of the present disclosure advantageously provide a solid or semi-solid convenient and pleasant dosage form for delivery of a combination of antiacid ingredient such as tricalcium phosphate, and one or more relaxing ingredient. In certain embodiments, the one or more relaxing ingredient can be present in the gummy dosage form in an amount of from about 10 mg to about 2 grams, about 50 mg to about 1 gram, about 50 mg to about 2 grams, about 100 mg to about 1 gram, or about 100 mg to about 500 mg.

Gummy dosage forms of the present disclosure advantageously provide a solid or semi-solid convenient and pleasant dosage form for delivery of a combination of antiacid ingredient such as tricalcium phosphate, and a relaxing ingredient such as Lemon balm extract, L-theanine, GABA or mixtures thereof. In certain embodiments, the one or more relaxing ingredient can be taste masked. Taste masking technologies are well known for a person skilled in the art.

The gummy dosage forms can include tricalcium phosphate, Lemon balm extract, L-theanine, GABA, one or more gelling agents, one or more acidulants, one or more sweeteners, one or more additional natural or herbal ingredients, water, or combinations thereof.

### Tricalcium Phosphate

In certain embodiments, the gummy dosage form can include tricalcium phosphate (Ca₃(PO₄)₂). The tricalcium phosphate can be incorporated in the form of a powder. In certain embodiments, tricalcium phosphate can be present in the gummy dosage form in an amount of from about 100 mg to about 2 g, about 200 mg to about 1.5 g, about 200 mg to about 500 mg, about 300 mg to about 350 mg, about 400 mg to about 900 mg, about 450 mg to about 850 mg, about 500 mg to about 800 mg, about 550 mg to about 750 mg, or about 600 mg to about 700 mg.

In certain embodiments, tricalcium phosphate can be present in the gummy dosage form in an amount of about 200 mg, about 250 mg, about 300 mg, about 350 mg, about 400 mg, about 500 mg, about 600 mg, about 650 mg, about 700 mg, about 800 mg, about 900 mg, about 1 gram, about 1.2 grams, about 1.4 grams, about 1.6 grams, about 1.8 grams, or about 2 grams.

In certain embodiments, elemental calcium sourced from tricalcium phosphate can be present in the gummy dosage form in an amount of about 50 mg, about 100 mg, about 150 mg, about 200 mg, about 250 mg, about 300 mg, about 350 mg, about 400 mg, about 450 mg, about 500 mg, about 600 mg, about 700 mg, or about 800 mg.

In certain embodiments, elemental calcium sourced from tricalcium phosphate can be present in the gummy dosage form in an amount of from about 30 mg to about 800 mg, about 40 mg to about 700 mg, about 50 mg to about 500 mg, about 100 mg to about 450 mg, about 100 mg to about 150 mg, about 150 mg to about 400 mg, about 200 mg to about 350 mg, or about 200 mg to about 300 mg.

In certain embodiments, tricalcium phosphate can be present in an amount of from about 1% to about 25%, about 5% to about 20%, about 6% to about 19%, about 7% to about 18%, about 8% to about 17%, about 9% to about 16%, or about 10% to about 15% by weight, based on the total weight of the gummy dosage form.

In certain embodiments, tricalcium phosphate can be present in an amount of from about 6%, about 7%, about 8%, about 9%, about 10%, about 11%, about 12%, about 13%, about 14%, about 15%, about 16%, about 17%, or about 18% by weight, based on the total weight of the gummy dosage form.

### Lemon Balm Extract

In certain embodiments, the gummy dosage form can include Lemon balm extract. The Lemon balm extract can be incorporated in the form of a liquid extract or a powder. In certain embodiments, the Lemon balm extract can contain at least about 3% rosmarinic acid, at least about 4% rosmarinic acid, at least about 5% rosmarinic acid, at least about 6% rosmarinic acid, at least about 7% rosmarinic acid, at least about 8% rosmarinic acid, at least about 10% rosmarinic acid, at least about 12% rosmarinic acid, or at least about 15% rosmarinic acid.

In certain embodiments, Lemon balm extract can be present in the gummy dosage form in an amount of from about 10 mg to about 2 grams, about 50 mg to about 1 gram, about 50 mg to about 2 grams, about 50 mg to about 500 mg, about 50 mg to about 200 mg, about 50 mg to about 100 mg, about 100 mg to about 1 gram, or about 100 mg to about 500 mg.

In certain embodiments, the gummy dosage form can include Lemon balm extract in an amount of about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1 gram, about 1.5 grams, or about 2 grams.

In certain embodiments, the Lemon balm extract can be present in an amount of from about 0.01% to about 20%, about 0.1% to about 20%, about 0.5% to about 15%, about 0.5% to about 10%, about 1% to about 20%, about 1% to about 15%, about 1% to about 10%, about 2% to about 15%, or about 2% to about 10% by weight, based on the total weight of the gummy dosage form.

In certain embodiments, the Lemon balm extract can be present in an amount of about 0.01%, about 0.1%, about 0.5%, about 0.6%, about 0.8%, about 1%, about 1.5%, about 2%, about 3%, about 4%, about 5%, about 6%, about 8%, or about 10% by weight, based on the total weight of the gummy dosage form.

In certain embodiments, the gummy dosage form can include at least about 0.1%, at least about 0.2%, at least about 0.4%, at least about 0.5%, at least about 0.6%, at least about 0.8%, at least about 1%, at least about 2%, or at least about 5% by weight of Lemon balm extract, based on the total weight of the gummy dosage form.

### L-Theanine

In certain embodiments, the gummy dosage form can include L-theanine. The L-theanine can be incorporated in the form of a liquid extract or a powder.

In certain embodiments, L-theanine can be present in the gummy dosage form in an amount of from about 10 mg to about 2 grams, about 50 mg to about 1 gram, about 50 mg to about 150 mg, about 50 mg to about 500 mg, or about 100 mg to about 300 mg.

In certain embodiments, the gummy dosage form can include L-theanine in an amount of about 10 mg, about 25 mg, about 50 mg, about 100 mg, about 150 mg, about 200 mg, about 250 mg, about 300 mg, about 500 mg, about 800 mg, or about 1 gram.

In certain embodiments, the L-theanine can be present in an amount of from about 0.01% to about 20%, about 0.1% to about 20%, about 0.2% to about 15%, about 0.5% to about 10%, about 0.5% to about 5%, about 1% to about 10%, or about 1% to about 5% by weight, based on the total weight of the gummy dosage form.

In certain embodiments, the L-theanine can be present in an amount of about 0.01%, about 0.1%, about 0.5%, about 0.6%, about 0.8%, about 1%, about 1.5%, about 2%, about 3%, about 4%, about 5%, about 8%, or about 10% by weight, based on the total weight of the gummy dosage form.

In certain embodiments, the gummy dosage form can include at least about 0.1%, at least about 0.2%, at least about 0.4%, at least about 0.5%, at least about 0.6%, at least about 0.8%, at least about 1%, or at least about 2% by weight of L-theanine, based on the total weight of the gummy dosage form.

### GABA

In certain embodiments, the gummy dosage form can include GABA. The GABA can be incorporated in the form of a liquid or a powder.

In certain embodiments, GABA can be present in the gummy dosage form in an amount of from about 10 mg to about 800 mg, about 30 mg to about 700 mg, about 30 mg to about 300 mg, about 30 mg to about 80 mg, about 50 mg to about 600 mg, about 50 mg to about 400 mg, about 50 mg to about 200 mg, about 70 mg to about 200 mg, or about 70 mg to about 150 mg.

In certain embodiments, the gummy dosage form can include GABA in an amount of about 10 mg, about 20 mg, about 40 mg, about 50 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 grams.

In certain embodiments, the GABA can be present in an amount of from about 0.01% to about 20%, about 0.1% to about 20%, about 0.5% to about 15%, about 0.5% to about 10%, about 1% to about 20%, about 1% to about 15%, about 1% to about 10%, or about 1% to about 5% by weight, based on the total weight of the gummy dosage form.

In certain embodiments, the GABA can be present in an amount of about 0.01%, about 0.1%, about 0.5%, about 0.6%, about 0.8%, about 1%, about 1.5%, about 2%, about 5%, about 8%, about 10%, about 12%, about 14%, about 16%, about 18%, or about 20% by weight, based on the total weight of the gummy dosage form.

In certain embodiments, the gummy dosage form can include at least about 0.1%, at least about 0.2%, at least about 0.4%, at least about 0.5%, at least about 0.6%, at least about 0.8%, at least about 1%, or at least about 1.5% by weight of GABA, based on the total weight of the gummy dosage form.

### Mixtures of Relaxing Ingredient

In certain embodiments, the gummy dosage form can include two relaxing ingredients. For example, the gummy dosage form can include Lemon balm extract and L-theanine, or Lemon balm extract and GABA, or L-theanine and GABA. The two relaxing ingredients can be present in ratios of 1:1, 1:2, 1:3, 1:4, 1:5, 1:6, 1:7, 1:8, 1:9, 1:10, 1:15, 1:20, 1:30, 1:40, 1:50, 1:60, 1:70, 1:80, 1:90, 1:100.

In certain embodiments, the gummy dosage form can include three relaxing ingredients. For example, the gummy dosage form can include Lemon balm extract and L-theanine and GABA.

### Gelling Agents

In certain embodiments, the gummy dosage form can include one or more gelling agents. A person skilled in the art will appreciate a wide variety of gelling agents are suitable for use with the present disclosure. Suitable gelling agents can include, but are not limited to, pectin, starches, celluloses, derived starches, gelatin, carrageenan, alginate, gums, and combinations thereof. In certain embodiments, the one or more gelling agents can include gelatin, pectin, kappa carrageenan, iota carrageenan, a combination of kappa and iota carrageenan, or combinations thereof.

In certain embodiments, the one or more gelling agents can include pectin. Suitable pectin may be low methoxylated pectin or high methoxylated pectin. A person skilled in the art will appreciate a wide variety of gums are suitable for use with the present disclosure. Suitable gums include gellan gum, xanthan gum, guar gum, locust bean gum, tragacanth, acacia gum, and combinations thereof. In certain embodiments, more than one gelling agent can be used. In certain embodiments, the gelling matrix can be created when the one or more gelling agents are dissolved into hot water, other ingredients are added, and the form is set in a mold and allowed to cool.

In certain embodiments, the one or more gelling agents can include a natural fiber, such as a pectin, purified from citrus peel.

In certain embodiments, the gummy dosage form is substantially free of gelatin.

The gummy dosage form can include the one or more gelling agents in an amount of from about 0.05% to about 10%, about 1% to about 8%, about 3% to about 5%, or about 3.5% to about 4% by weight, based on the total weight of the gummy dosage form. In certain embodiments, the gummy dosage form can include the one or more gelling agents in an amount of about 0.05%, about 0.1%, about 0.5%, about 1%, about 3%, about 3.5%, about 4%, about 5%, about 8%, or about 10% by weight, based on the total weight of the gummy dosage form.

### Acidulants

In certain embodiments, the gummy dosage form can include one or more acidulants. A person skilled in the art will appreciate a wide variety of acidulants are suitable for use with the present disclosure. Suitable acidulants include, for example, and not by way of limitation, organic acids such as citric acid, malic acid, fumaric acid, or combinations thereof. In certain embodiments, the one or more acidulants can include citric acid, sodium citrate, or combinations thereof.

In certain embodiments, the gummy dosage form can include the one or more acidulants present in an amount of from about 0.1% to about 10%, about 0.1% to about 5%, about 0.5% to about 3%, about 0.5% to about 2%, or about 1% to about 1.5% by weight, based on the total weight of the gummy dosage form. In certain embodiments, the gummy dosage form can include the one or more acidulants in an amount of about 0.1%, about 0.5%, about 1%, about 1.2%, about 1.5%, about 2%, about 2.5%, about 3%, about 4%, about 5%, about 8%, or about 10% by weight, based on the total weight of the gummy dosage form.

### Sweeteners

In certain embodiments, the gummy dosage form can include one or more sweeteners. A person skilled in the art will appreciate a wide variety of sweeteners are suitable for use with the present disclosure. Suitable sweeteners can include, for example, and not by way of limitation, synthetic or natural sugars; sugars extracted from sugar cane and sugar beet (sucrose), dextrose (also called glucose), fructose (also called laevulose), and lactose (also called milk sugar); isomalt; corn syrup, high fructose corn syrup, stevia, and mixtures thereof. In certain embodiments, the gummy dosage form can include one or more sweeteners selected from the group consisting of glycose syrup, stevia Rebaudioside A, Stevia leaf extract, monk fruit, sucrose, fructose, tapioca syrup, dextrose, and combinations thereof.

In certain embodiments, the one or more sweeteners can include sucrose, glucose syrup, or combinations thereof.

In certain embodiments, the gummy dosage form is substantially free of high fructose corn syrup. In certain embodiments, the gummy dosage form is substantially free of artificial sweeteners.

In certain embodiments, the gummy dosage form can include the one or more sweeteners in an amount of from about 0.02% to about 90%, about 0.5% to about 90%, about 5% to about 75%, about 10% to about 50%, about 50% to about 90%, about 40% to about 80%, or about 50% to about 75% by weight, based on the total weight of the gummy dosage form. In particular embodiments, the gummy dosage form can include the one or more sweeteners in an amount of about 0.02%, about 0.5%, about 5%, about 10%, about 15%, about 20%, about 25%, about 30%, about 35%, about 40%, about 45%, about 50%, about 55%, about 60%, about 65%, about 70%, about 74% about 75%, about 80%, about 85%, or about 90% by weight, based on the total weight of the gummy dosage form. In certain embodiments, the gummy dosage form can include the one or more sweeteners in an amount of at least about 15%, at least about 25%, at least about 40%, at least about 50%, at least about 60%, or at least about 70% by weight, based on the total weight of the gummy dosage form.

In certain embodiments, the one or more sweeteners included in the gummy dosage form can be a high intensity sweetener, for example, stevia, sucralose, or neotame. In such embodiments, the gummy dosage form can include the one or more sweeteners in an amount of from about 0.02% to about 3%, about 0.1% to about 1%, about 0.5% to about 3%, about 0.5% to about 2.5%, or about 1% to about 2% by weight, based on the total weight of the gummy dosage form. In particular embodiments, the gummy dosage form can include the one or more sweeteners in an amount of about 0.02%, about 0.05%, about 0.1%, about 0.2%, about 0.5%, about 0.8%, about 1%, about 1.5%, about 2%, about 2.5%, or about 3% by weight, based on the total weight of the gummy dosage form.

In certain embodiments the one or more sweeteners included in the gummy dosage form can be a bulk sweetener, for example, dextrose. In such embodiments, the gummy dosage form can include the one or more sweeteners in an amount of from about 10% to about 90%, about 15% to about 75%, about 25% to about 50%, about 50% to about 90%, or about 75% to about 90% by weight, based on the total weight of the gummy dosage form. In particular embodiments, the gummy dosage form can include the one or more sweeteners in an amount of about 10%, about 15%, about 20%, about 25%, about 30%, about 40%, about 45%, about 50%, about 55%, about 60%, about 65%, about 70%, about 75%, about 80%, about 85%, or about 90% by weight, based on the total weight of the gummy dosage form.

In certain embodiments, the gummy dosage form can include more than one sweetener. In such embodiments, the gummy dosage form can include at least a first sweetener in an amount of from about 10% to about 50%, about 20% to about 40%, or about 25% to about 35% by weight, based on the total weight of the gummy dosage form. In certain embodiments, the gummy dosage form can further include a second sweetener in an amount of from about 25% to about 65%, about 30% to about 50%, or about 35% to about 45% by weight, based on the total weight of the gummy dosage form.

### Additional Natural or Herbal Ingredients

In certain embodiments, the gummy dosage form can include one or more additional natural or herbal ingredients. A person skilled in the art will appreciate a wide variety of additional natural or herbal ingredients are suitable for use with the present disclosure.

In certain embodiments, such ingredients can be complimentary for the purposes of sleep, for example, and not by way of limitation, including melatonin, chamomile, valerian root, or combinations thereof. In certain embodiments, the one or more additional natural or herbal ingredients can include zinc, melatonin, Vitamin C (ascorbic acid), elderberry, thyme, or combinations thereof.

In certain embodiments, the gummy dosage form can include the one or more additional natural or herbal ingredients in an amount of from about 0.1% to about 40%, about 1% to about 30%, about 5% to about 25%, or about 10% to about 20% by weight, based on the total weight of the gummy dosage form. In certain embodiments, the gummy dosage form can include the one or more additional natural or herbal ingredients in an amount of about 0.1%, about 0.5%, about 1%, about 5%, about 10%, about 11%, about 15%, about 20%, about 25%, about 30%, about 35%, or about 40% by weight, based on the total weight of the gummy dosage form. A person skilled in the art will appreciate an amount of the one or more additional natural or herbal ingredients can widely vary, for example, depending on the actual dose.

### Water

In certain embodiments, the gummy dosage form can include water. In certain embodiments, the gummy dosage form can include from about 1% to about 20%, about 5% to about 15%, or about 5% to about 10% by weight water, based on the total weight of the gummy dosage form. In certain embodiments, the gummy dosage form can include about 1%, about 5%, about 8%, about 9%, or about 10% by weight water, based on the total weight of the gummy dosage form.

### Gummy Dosage Form Formulations

In certain embodiments, the gummy dosage form can include tricalcium phosphate, Lemon balm extract, one or more gelling agents, one or more acidulants, one or more sweeteners, one or more additional natural or herbal ingredients, water, or combinations thereof.

In certain embodiments, the gummy dosage form can include tricalcium phosphate in an amount of from about 1% to about 25% by weight; Lemon balm extract in an amount of from about 0.01% to about 20% by weight; one or more gelling agents in an amount of from about 0.05% to about 10% by weight; one or more acidulants in an amount of from about 0.1% to about 10% by weight; one or more sweeteners in amount of from about 0.02% to about 90% by weight; one or more additional natural or herbal ingredients in an amount of from about 0.1% to about 40% by weight; and water in an amount of from about 1% to about 20% by weight, each based on the total weight of the gummy dosage form. The one or more gelling agents can include pectin. The one or more acidulants can include citric acid (e.g., in a 50% solution with purified water), sodium citrate, or combinations thereof.

In certain embodiments, the gummy dosage form can include tricalcium phosphate, L-theanine, one or more gelling agents, one or more acidulants, one or more sweeteners, one or more additional natural or herbal ingredients, water, or combinations thereof.

In certain embodiments, the gummy dosage form can include tricalcium phosphate in an amount of from about 1% to about 25% by weight; L-theanine in an amount of from about 0.01% to about 20% by weight; one or more gelling agents in an amount of from about 0.05% to about 10% by weight; one or more acidulants in an amount of from about 0.1% to about 10% by weight; one or more sweeteners in amount of from about 0.02% to about 90% by weight; one or more additional natural or herbal ingredients in an amount of from about 0.1% to about 40% by weight; and water in an amount of from about 1% to about 20% by weight, each based on the total weight of the gummy dosage form. The one or more gelling agents can include pectin. The one or more acidulants can include citric acid (e.g., in a 50% solution with purified water), sodium citrate, or combinations thereof.

In certain embodiments, the gummy dosage form can include tricalcium phosphate, GABA, one or more gelling agents, one or more acidulants, one or more sweeteners, one or more additional natural or herbal ingredients, water, or combinations thereof.

In certain embodiments, the gummy dosage form can include tricalcium phosphate in an amount of from about 1% to about 25% by weight; GABA in an amount of from about 0.01% to about 20% by weight; one or more gelling agents in an amount of from about 0.05% to about 10% by weight; one or more acidulants in an amount of from about 0.1% to about 10% by weight; one or more sweeteners in amount of from about 0.02% to about 90% by weight; one or more additional natural or herbal ingredients in an amount of from about 0.1% to about 40% by weight; and water in an amount of from about 1% to about 20% by weight, each based on the total weight of the gummy dosage form. The one or more gelling agents can include pectin. The one or more acidulants can include citric acid (e.g., in a 50% solution with purified water), sodium citrate, or combinations thereof.

In certain embodiments, the gummy dosage form can be substantially free of one or more of high fructose corn syrup, artificial sweeteners, artificial flavors, gluten, or gelatin. In certain embodiments, the gummy dosage form can be substantially free of high fructose corn syrup, artificial sweeteners, artificial flavors, gluten, and gelatin. In certain embodiments, the gummy dosage form can be substantially free of an artificial sweetener.

### Coating

In certain embodiments, the gummy dosage form of the present disclosure can include a coating. The coating can be disposed on at least a portion of the gummy dosage form. In certain embodiments, the coating can wholly encompass the gummy dosage form. The coating can be disposed on gummy dosage forms of the present disclosure by various suitable means as known by those skilled in the art. In certain embodiments, the coating can be homogeneous. The coating can be present and applied in an amount of from about 0.5% to about 10%, about 0.5% to about 8%, about 1% to about 5%, or about 1% to about 3% by weight, based on the total weight of the gummy dosage form. In particular embodiments, the coating can be present and applied in an amount of about 0.5%, about 1%, about 2%, about 3%, about 4%, about 5%, about 8%, or about 10% by weight, based on the total weight of the gummy dosage form.

The coating can include various ingredients as known in the art. In certain embodiments, the coating can include one or more sugars, or one or more sugar alcohols. For example, and not by way of limitation, materials suitable for sugar or sugar alcohol-based coatings can include sucrose, dextrose, xylitol, or combinations thereof. In certain embodiments, the coating can be a film coating including one or more polymers. For example, and not by way of limitation, polymers suitable for film coatings can include hypromellose and polyvinyl alcohol, polyvinyl alcohol:polyethylene glycol co-polymers, and mixtures thereof. In certain embodiments, the coating can be a film coating including one or more waxes. For example, carnuba wax and/or a combination of carnuba wax dispersed in vegetable oil, like refined coconut oil such as medium chain triglyceride (MCT).

### Additional Additives

Gummy dosage forms of the presently disclosed subject matter can include additional additives. For example, the gummy dosage form can include one or more of gelling agents, fats, proteins, colorings, flavorings, acidulants, thickeners, emulsifiers, antioxidants, preservatives, lubricants, glidants, disintegrants, or combinations thereof.

### Flavorings

In certain embodiments, the gummy dosage form can include one or more flavorings. A person skilled in the art will appreciate a wide variety of flavorings are suitable for use with the present disclosure. The one or more flavorings can include any suitable flavorings, for example, and not by way of limitation, peppermint, spearmint, eucalyptus, licorice, vanilla, caramel, mixed berries, mixed fruits, black current, blueberry, cherry, lemon, strawberry, orange or combinations thereof.

In certain embodiments, the gummy dosage form can include the one or more flavorings in an amount of from about 0.01% to about 5%, about 0.01% to about 3%, about 0.1% to about 3%, about 0.5% to about 1.5%, or about 1% to about 3% by weight, based on the total weight of the gummy dosage form. In particular embodiments, the gummy dosage form can include the one or more flavorings in an amount of about 0.01%, about 0.05%, about 0.08%, about 0.1%, about 0.5%, about 0.8%, about 1%, about 1.5%, about 2%, about 2.5%, about 3%, about 3.5%, about 4%, about 4.5%, or about 5% by weight, based on the total weight of the gummy dosage form.

### Acidulants

In certain embodiments, the gummy dosage form can include one or more acidulants. A person skilled in the art will appreciate a wide variety of acidulants are suitable for use with the present disclosure. Suitable acidulants can include, for example, and not by way of limitation, organic acids such as citric acid, malic acid, fumaric acid, or combinations thereof. In certain embodiments, the one or more acidulants can include citric acid, sodium citrate, or combinations thereof.

In certain embodiments, the gummy dosage form can include the one or more acidulants present in an amount of from about 0.1% to about 10%, about 0.1% to about 5%, about 0.5% to about 3%, about 0.5% to about 2%, or about 1% to about 1.5% by weight, based on the total weight of the gummy dosage form. In certain embodiments, the gummy dosage form can include the one or more acidulants in an amount of about 0.1%, about 0.5%, about 1%, about 1.2%, about 1.5%, about 2%, about 2.5%, about 3%, about 4%, about 5%, about 8%, or about 10% by weight, based on the total weight of the gummy dosage form.

### Solvents

In certain embodiments, the gummy dosage form can include one or more solvents. A person skilled in the art will appreciate a wide variety of solvents are suitable for use with the present disclosure. Suitable solvents can include, for example, and not by way of limitation, propylene glycol, polyethylene glycol, glycerin, or combinations thereof.

In certain embodiments, the gummy dosage form can include the one or more solvents in an amount of from about 2% to about 30%, about 5% to about 25%, about 10% to about 30% or about 5% to about 15% by weight, based on the total weight of the gummy dosage form.

### Excipients

In certain embodiments, the gummy dosage form can include one or more excipients. A person skilled in the art will appreciate a wide variety of excipients are suitable for use with the present disclosure. Suitable excipients can include, for example, and not by way of limitation, fats, proteins, fillers, glidants, lubricants, sweeteners, flavors, coloring agents, fillers, binding/gelling agents, and mixtures thereof.

In certain embodiments, the gummy dosage form can include the one or more excipients in an amount of from about 50% to about 99%, about 60% to about 95%, about 70% to about 99%, about 80% to about 99%, or about 85% to about 95% by weight, based on the total weight of the gummy dosage form.

### Lubricants

In certain embodiments, the gummy dosage form can include one or more lubricants. A person skilled in the art will appreciate a wide variety of lubricants are suitable for use with the present disclosure. Suitable lubricants can include, for example, and not by way of limitation, long chain fatty acids and their salts, such as magnesium stearate and stearic acid, talc, glycerides waxes, and mixtures thereof.

### Glidants

In certain embodiments, the gummy dosage form can include one or more glidants. A person skilled in the art will appreciate a wide variety of glidants are suitable for use with the present disclosure. Suitable glidants can include, for example, and not by way of limitation, colloidal silicon dioxide.

In certain embodiments, the gummy dosage form can include the one or more glidants in an amount of from about 0.1% to about 3%, about 0.5% to about 2.5%, about 1% to about 3%, or about 1.5% to about 2.5% by weight, based on the total weight of the gummy dosage form. In particular embodiments, the gummy dosage form can include the one or more glidants in an amount of about 0.1%, about 0.5%, about 1%, about 1.5%, about 2%, about 2.5%, or about 3% by weight, based on the total weight of the gummy dosage form.

### Gelling Agents

In certain embodiments, the gummy dosage form can include one or more gelling agents. A person skilled in the art will appreciate a wide variety of gelling agents are suitable for use with the present disclosure. Suitable gelling agents can include, but are not limited to, pectin, starches, celluloses, derived starches, gelatin, carrageenan, alginate, gums, and combinations thereof. In certain embodiments, the one or more gelling agents can include gelatin, pectin, kappa carrageenan, iota carrageenan, a combination of kappa and iota carrageenan, or combinations thereof. In certain embodiments, the one or more gelling agents can include pectin. A person skilled in the art will appreciate a wide variety of gums are suitable for use with the present disclosure. Suitable gums can include gellan gum, xanthan gum, guar gum, locust bean gum, tragacanth, acacia gum, and combinations thereof.

In certain embodiments, the gummy dosage form can include the one or more gelling agents in an amount of from about 0.5% to about 30%, about 1% to about 25%, or about 5% to about 15% by weight, based on the total weight of the gummy dosage form.

In particular embodiments, the gummy dosage form can include the one or more gelling agents in an amount of about 0.5%, about 1%, about 5%, about 10%, about 15%, about 20%, about 25%, or about 30% by weight, based on the total weight of the gummy dosage form.

### Coloring Agents

In certain embodiments, the gummy dosage form can include one or more coloring agents. A person skilled in the art will appreciate a wide variety of coloring agents are suitable for use with the present disclosure. Suitable coloring agents can include, for example, and not by way of limitation, lakes and dyes approved as a food additive, including natural coloring agents, and combinations thereof. In certain embodiments the gummy dosage form can include one or more coloring agents selected from Maqui Berry juice concentrate, Purple Carrot juice concentrate, Annatto, and combinations thereof.

In certain embodiments, the gummy dosage form can include the one or more coloring agents in an amount of from about 0.01% to about 5%, about 0.05% to about 3%, about 0.5% to about 2%, or about 1% to about 3% by weight, based on the total weight of the gummy dosage form. In particular embodiments, the gummy dosage form can include the one or more coloring agents in an amount of about 0.01%, about 0.05%, about 0.1%, about 0.5%, about 1%, about 1.5%, about 2%, about 3%, about 4%, or about 5% by weight, based on the total weight of the gummy dosage form.

### Fillers

In certain embodiments, the gummy dosage form can include one or more fillers. A person skilled in the art will appreciate a wide variety of fillers are suitable for use with the present disclosure. Suitable fillers include corn syrup, sucrose, starches, fats, proteins, gelatin, or combinations thereof. Additional materials that can be used in the gummy dosage form as fillers include, for example, corn syrup solids, sucrose, starches, fats, coconut oil, lecithin, proteins, gelatin, or combinations thereof.

In certain embodiments, the gummy dosage form can include the one or more fillers in an amount of from about 30% to about 95%, about 40% to about 80%, about 35% to about 75%, or about 50% to about 65% by weight, based on the total weight of the gummy dosage form. In particular embodiments, the gummy dosage form can include the one or more fillers in an amount of about 30%, about 35%, about 40%, about 45%, about 50%, about 55%, about 60%, about 65%, about 70%, about 75%, about 80%, about 85%, about 90%, or about 95% by weight, based on the total weight of the gummy dosage form.

### Fats

In certain embodiments, the gummy dosage form can include one or more fats. A person skilled in the art will appreciate a wide variety of fats are suitable for use with the present disclosure.

In certain embodiments, the gummy dosage form can include one or more fats in an amount of from about 1% to about 30%, about 10% to about 30%, about 5% to about 20%, or about 10% to about 15% by weight, based on the total weight of the gummy dosage form.

### Proteins

In certain embodiments, the gummy dosage form can include one or more proteins. A person skilled in the art will appreciate a wide variety of proteins are suitable for use with the present disclosure.

In certain embodiments, the gummy dosage form can include one or more proteins in an amount of from about 1% to about 20%, about 5% to about 15%, or about 10% to about 20% by weight, based on the total weight of the gummy dosage form. In particular embodiments, the gummy dosage form can include one or more proteins in an amount of about 1%, about 5%, about 8%, about 10%, about 15%, or about 20% by weight, based on the total weight of the gummy dosage form.

### Methods of Making Gummy Dosage Forms

In certain embodiments, a method of manufacturing a gummy dosage form of the present disclosure is provided. A wide variety of processes can be used to provide the gummy dosage form of the present disclosure. In certain embodiments, the gummy dosage form can be produced using a starch mold process in which a bed of starch is pre-molded prior to the deposition of a flowable gummy dosage form formulation. The flowable gummy dosage form formulation is allowed to form and cool. Alternatively, the gummy dosage form can be produced with a non-starch mold, such as a ceramic, silicone, metal, or coated metal mold, in which the flowable gummy dosage form formulation is directly deposited in the mold and cooled to form the gummy dosage form.

The gummy dosage form can be prepared by mixing and heating a solution and depositing the heated solution into a mold. The flowable gummy dosage form formulation is allowed to cool to room temperature or approximately 25° C.

In certain embodiments, a method of manufacturing a gummy dosage form of the present disclosure is provided. The method can include preparation of gummy dosage form. In certain embodiments, the gelling matrix of the gummy dosage form can be created when the one or more gelling agents are dissolved into hot water, other ingredients are added, and the form is set in a mold and allowed to cool. The gummy dosage form can be prepared, for example, by forming a mixture of a gelling agent, a first acidulant, and a first portion of a first sweetener (e.g., one third), which can be provided as dry ingredients. The dry mixture can be added to water (90° C. to 105° C.) to form a solution and stirred until the gelling agent swells. The solution can be brought to a boil to form a boiling mixture. A second portion of the first sweetener (e.g., two thirds) and a second acidulant mixed with purified water to create a 50% solution can both be added to the boiling mixture and mixed until dissolved. In a separate container, a second sweetener can be brought to a boil and added to the other mixture and mixed. A relaxing ingredient selected from Lemon balm extract, L-theanine, GABA or mixtures thereof; and one or more additional natural or herbal ingredients can be added to the mixture and mixed until dissolved. Preferably the relaxing ingredient selected from Lemon balm extract, L-theanine, GABA or mixtures thereof, and the tricalcium phosphate are added to the mixture before the acidulant. Tricalcium phosphate may be added in two portions, for example a third and two thirds. The resulting mixture can be added to molds and allowed to cool to form the gummy dosage form. In certain embodiments, the gelling agent can be pectin, the first acidulant can be sodium citrate, the second acidulant can be citric acid, the first sweetener can be sucrose, and the second sweetener can be glucose syrup.

### Methods of Using Gummy Dosage Forms

Gummy dosage forms of the present disclosure can be used, for example, alone as a dosage form for delivery of one or more therapeutic ingredients. The gummy dosage forms of the present disclosure can be administered orally. Methods of treatment of a subject suffering from a gastric disease or disorder such as dyspepsia, abdominal fullness, heartburn, nausea, belching, or upper abdominal pain, by use of the gummy dosage forms described herein are provided. In certain embodiments, the subject can be suffering from a heartburn which is due to gastroesophageal reflux disease (GERD) or gastritis.

In certain embodiments, the subject can be suffering from difficulty sleeping from heartburn and the gummy dosage form can include a relaxing ingredient.

In certain embodiments, the subject can be suffering from stress induced heartburn and the gummy dosage form can include a relaxing ingredient.

### Features of Gummy Dosage Forms

The present disclosure provides for gummy dosage forms. Such gummy dosage forms can be semi-solid or solid.

In certain embodiments, the gummy dosage form can be of a weight which can accommodate a portion of tricalcium phosphate and a portion of a relaxing ingredient selected from Lemon balm extract, L-theanine, GABA or mixtures thereof. The gummy dosage form can have a mass of from about 0.5 grams to about 10 grams, about 1 gram to about 8 grams, or about 2 grams to about 6 grams. In certain embodiments, the gummy dosage form can have a mass of about 1 gram to about 5 grams, about 1 gram to about 5.5 grams, about 1 gram to about 6 grams, about 1 gram to about 6.5 grams, about 1 gram to about 7 grams, about 1 gram to about 7.5 grams, about 1 gram to about 8 grams, about 1.5 grams to about 5 grams, about 1.5 grams to about 5.5 grams, about 1.5 grams to about 6 grams, about 1.5 grams to about 6.5 grams, about 1.5 grams to about 7 grams, about 1.5 grams to about 7.5 grams, about 1.5 grams to about 8 grams, about 2 grams to about 5 grams, about 2 grams to about 5.5 grams, about 2 grams to about 6 grams, about 2 grams to about 6.5 grams, about 2 grams to about 7 grams, about 2 grams to about 7.5 grams, about 2 grams to about 8 grams, about 2.5 grams to about 5 grams, about 2.5 grams to about 5.5 grams, about 2.5 grams to about 6 grams, about 2.5 grams to about 6.5 grams, about 2.5 grams to about 7 grams, about 2.5 grams to about 7.5 grams, about 2.5 grams to about 8 grams, about 3 grams to about 5 grams, about 3 grams to about 5.5 grams, about 3 grams to about 6 grams, about 3 grams to about 6.5 grams, about 3 grams to about 7 grams, about 3 grams to about 7.5 grams, about 3 grams to about 8 grams, about 3.5 grams to about 5 grams, about 3.5 grams to about 5.5 grams, about 3.5 grams to about 6 grams, about 3.5 grams to about 6.5 grams, about 3.5 grams to about 7 grams, about 3.5 grams to about 7.5 grams, about 3.5 grams to about 8 grams, about 4 grams to about 5 grams, about 4 grams to about 5.5 grams, about 4 grams to about 6 grams, about 4 grams to about 6.5 grams, about 4 grams to about 7 grams, about 4 grams to about 7.5 grams, about 4 grams to about 8 grams, about 4.5 grams to about 5 grams, about 4.5 grams to about 5.5 grams, about 4.5 grams to about 6 grams, about 4.5 grams to about 6.5 grams, about 4.5 grams to about 7 grams, about 4.5 grams to about 7.5 grams, about 4.5 grams to about 8 grams, about 5 grams to about 5.5 grams, about 5 grams to about 6 grams, about 5 grams to about 6.5 grams, about 5 grams to about 7 grams, about 5 grams to about 7.5 grams, or about 5 grams to about 8 grams. In particular embodiments, the gummy dosage form can have a mass of about 4 grams, about 4.5 grams, about 5 grams, about 5.5 grams, about 6 grams, about 6.5 grams, or about 7 grams.

### Other Dosage Forms

Compositions of the present disclosure can be utilized in other dosage forms. Such forms include, for example, lozenges, chewing gums, and soft chewables (“soft chews”).

Lozenges can have a candy glass or compressed outer shell portion and are intended to dissolve in the oral cavity such as the mouth or pharynx without chewing. For lozenges, the dissolved materials from the dosage form are eventually ingested.

Soft chew forms are intended as dosage forms which retain a portion of their integrity and texture upon chewing, after which they are swallowed. Soft chews can be opaque and contain at least one of corn starch, corn syrup, corn syrup solids, glycerin, coconut oil, palm oil, or lecithin. Soft chews can be differentiated from gummy dosage forms, for example, through the incorporation of a lipid such as palm oil or coconut oil.

Chewing gum forms are intended for chewing in the oral cavity while the therapeutic ingredients contained in the form are released. Chewing gums are not intended to be swallowed. The shell of a chewing gum typically comprises a chewing gum base which further comprises resins and elastomers which are not digestible.

### EXAMPLES

The following Examples are intended to illustrate, but not to limit, the disclosed subject matter in any manner, shape, or form, either explicitly or implicitly.

### Prophetic Example 1: Gummy Dosage Form

### Gummy Dosage Form Preparation:

The following exemplary gummy dosage form preparation results in a dome shaped and approximately 5.5 grams sugar gummy. According to an embodiment, the following steps may be taken.

Step 1: The pectin (or replacement), optionally sodium citrate, and approximately one third (⅓) of the sucrose according to the Formula in Table 1 are added as dry components to a plastic bag and manually mixed.

Step 2: The citric acid is added to purified water and mixed to create a 50% solution according to the Formula in Table 1.

Step 3: The water according to the Formula in Table 1 is heated (90° C. to 105° C.) in a suitable container.

Step 4: The pectin (or replacement), optionally sodium citrate, and sucrose blend from Step 1 is added to the water from Step 3 and stirred manually until the pectin swells. The solution is brought to a boil.

Step 5: The remaining sucrose (approximately two thirds (⅔) of the sucrose according to the Formula in Table 1).

Step 6: The glucose syrup according to the Formula in Table 1 is placed into an additional container and brought to a boil, and then added to the mixture from Step 5, mixed and boiled (90° C. to 105° C.) to bring the solution to approximatively 78 Brix.

Step 7: The tricalcium phosphate, the Lemon balm extract (standardized to >6% rosmarinic acid), and optional natural colorant and natural flavor, according to the Formula in Table 1 are added to the mixture in Step 6 and mixed until dissolved.

Step 8: The citric acid/purified water mixture from Step 2 are added to the mixture in Step 7 to adjust the pH (typically 3.2 to 3.8, but depending on the pectin it may be higher, for example up to pH 5 for methoxylated pectin) and mixed until dissolved.

Step 9: The mixture in Step 8 is added to molds at a temperature not less than 95° C. and allowed to cool to form a 5.5 g gummy.

Natural colorant and natural flavor are optional and thus are not indicated in Table 1.

Examples of suitable natural colorants and natural flavors are provided in Table 2.

Example 1 provides a formulation with Lemon balm extract as relaxing ingredient; a person skilled in the art can replace the Lemon balm extract with GABA or L-theanine and adapt the proportions of sucrose, glucose syrup accordingly.

TABLE 1
Gummy Dosage Form Formula

| | Batch | mg/Gummy | % w/w for 5.5 g |
|---|---|---|---|
| Ingredient | Weight (g) | piece | Gummy |
| Pectin | 37.3 | 205.2 | 3.73 |
| Sodium Citrate | 2.8 | 15.4 | 0.28 |
| Sucrose | 289.7 | 1593.4 | 28.97 |
| Glucose Syrup | 422.2 | 2322.1 | 42.22 |
| Citric Acid | 9.3 | 51.2 | 0.93 |
| (as a 50% Solution) | | | |
| Lemon balm (Melissa | 27.3 | 150 | 2.73 |
| Officinalis) extract | | | |
| TriCalcium Phosphate | 118.2 | 650 | 11.82 |
| Water* | 265.6 (93.2) | 512.7 | 9.32 |
| TOTAL | 1000 g | 5500 mg | 100% w/w |

*A portion of water added during processing is removed upon boiling. 172.4 g removed (93.2 g remains)

TABLE 2
Example of natural colorants and natural flavors
in relation with the desired gummy color.

| Color | Natural Colorants | Natural Flavor |
|---|---|---|
| Purple | Maqui Berry Juice Concentrate | Cherry |
| | Purple Carrot Juice Concentrate | |
| Pink | Purple Carrot Juice Concentrate | Strawberry |
| Orange | Annatto | Orange |

### Example 2: Gummy Dosage Form

The gummy dosage forms disclosed in tables 3 and table 4 below were prepared according to the procedure disclosed in Example 1.

Ceamfibre 7240 and Ceampectin ESS 49092 are natural fiber ingredients purified from citrus peel; they were supplied by Ceamsa (Compania Espariola de Algas Marinas, S.A. Ponifio, Pontevedra, Spain). Ceamfibre 7240 and Ceampectin ESS 49092 were used a replacement of pectin in the step 1 of the process disclosed in Example 1.

Lemon Balm Extract 10:1 was supplied by Jiaherb; Shaanxi Jiahe Phytochem Co., Ltd., Xi'an, China. The Lemon Balm Extract had a rosmarinic acid content between 3% and 5%. 10:1 indicates that the herbal extract is 10 time concentrated (1 g of extract is equivalent to 10 g of the herb).

TABLE 3
Example of a Gummy Dosage Form Formula (Sample 1)

| | Batch | mg/Gummy piece | |
|---|---|---|---|
| | Weight (g) | (5000 mg) | % w/w |
| Ceamfibre 7240 | 20.4 | 102.00 | 2.04 |
| Trisodium Citrate | 4.8 | 24.00 | 0.48 |
| Citric Acid | 4.4 | 22.00 | 0.44 |
| Sucrose | 295 | 1475.00 | 29.50 |
| Purified Water * | 300 (67.5)* | 337.50 | 6.75 |
| Glucose Syrup (Corn | 396.8 | 1984.00 | 39.68 |
| Syrup DE = 42%) | | | |
| TriCalcium Phosphate | 129.4 | 647.00 | 12.94 |
| Color | 5 | 25.00 | 0.50 |
| Flavor | 20 | 100.00 | 2.00 |
| Citric Acid Solution | 19.2 | 96.00 | 1.92 |
| (50% w/w) | | | |
| Lemon Balm Extract | 37.5 | 18.5 | 3.75 |
| 10:1 | | | |
| TOTAL | 1000 g | 5000 mg | 100% w/w |

*A portion of water added during processing was removed upon boiling (67.5 g remained).

TABLE 4
Example of a Gummy Dosage Form Formula (Sample 2)

| | Batch | mg/Gummy piece | |
|---|---|---|---|
| | Weight (g) | (5000 mg) | % w/w |
| Ceampectin ESS 49092 | 20.4 | 102 | 2.04 |
| Sucrose | 211.6 | 1058 | 21.16 |
| Purified Water * | 250 (136.6)* | 683 | 13.66 |
| Glucose Syrup (Corn | 420 | 2100 | 42.00 |
| Syrup DE = 42%) | | | |
| TriCalcium Phosphate | 129.4 | 647 | 12.94 |
| Color | 4 | 20 | 0.40 |
| Flavor | 16 | 80 | 1.60 |
| Citric Acid Solution | 14 | 70 | 1.40 |
| (50% w/w) | | | |
| Lemon Balm Extract | 48 | 240 | 4.80 |
| 10:1 | | | |
| TOTAL | 1000 g | 5000 mg | 100% w/w |

*A portion of water added during processing was removed upon boiling (136.6 g remained).

It has been observed that about 20% of the Lemon Balm extract degrades during the manufacturing process. Therefore, the final amount of Lemon Balm extract per gummy piece can be estimated about 150 mg per gummy piece for Sample 1, and about 200 mg per gummy piece for Sample 2.

Gummy pieces according to Sample 1 and Sample 2 provided acceptable visual appearance (color, shape) and organoleptic properties (firmness, taste).

Sample 1 and Sample 2 disclose gummy pieces of 5000 mg. Production of smaller gummy pieces, such as 2000 mg, 2500 mg or 3000 mg for example, would reduce the content of tricalcium phosphate and Lemon Balm Extract per gummy piece. In order to maintain the same amount of tricalcium phosphate and Lemon Balm Extract per serving, it is possible to consider a serving comprising multiple gummy pieces, such as 2, 3 or 4 pieces. For example, a two gummy piece serving comprising 2500 mg gummy pieces. Small gummy piece may be easier to chew and to swallow.

While systems and methods have been described in connection with the various embodiments of the description, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention. Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

1. A gummy dosage form comprising tricalcium phosphate and a relaxing ingredient selected from Lemon balm extract, L-theanine, GABA, or mixtures thereof.

2. The gummy dosage form according to claim 1, wherein the relaxing ingredient is Lemon balm extract.

3. The gummy dosage form according to claim 2, wherein the gummy dosage form comprises Lemon balm extract in an amount of from about 10 mg to about 2 g.

4. The gummy dosage form according to claim 1, wherein the relaxing ingredient is L-theanine.

5. The gummy dosage form according to claim 4, wherein the gummy dosage form comprises L-theanine in an amount of from about 10 mg to about 2 g.

6. The gummy dosage form according to claim 1, wherein the relaxing ingredient is GABA.

7. The gummy dosage form according to claim 6, wherein the gummy dosage form comprises GABA in an amount of from about 10 mg to about 800 mg.

8. The gummy dosage form according to claim 1, wherein the gummy dosage form comprises tricalcium phosphate in an amount of from about 100 mg to about 2 g.

9. The gummy dosage form according to claim 1, wherein the gummy dosage form comprises sucrose.

10. The gummy dosage form according to claim 1, wherein the gummy dosage form is substantially free of one or more of high fructose corn syrup, artificial sweeteners, artificial flavors, gluten, or gelatin.

11. The gummy dosage form according to claim 1, wherein the gummy dosage form has a mass of from about 0.5 gram to about 10 grams.

12. The gummy dosage form according to claim 11, wherein the gummy dosage form has a mass of from about 4 grams to about 6 grams.

13. A method of making a gummy dosage form, comprising the steps of:
blending tricalcium phosphate and a relaxing ingredient in an aqueous pectin solution to form a blend;
pouring the blend in a mold; and
allowing the blend to cool in the mold to form the gummy dosage form, wherein the relaxing ingredient is selected from Lemon balm extract, L-theanine, GABA, or mixtures thereof.

14. A method of treating a gastric disease or disorder, comprising administering a gummy dosage form to a patient in need thereof, wherein the gummy dosage form comprises tricalcium phosphate and a relaxing ingredient selected from Lemon balm extract, L-theanine, GABA, or mixtures thereof.

15. A method according to claim 14, wherein difficulty of sleeping related to a gastric disease or disorder is treated.
